(19) United States
(12) Patent Application Publication
(10) Pub. No.: US 2023/0048130 A1
(43) Pub. Date: Feb. 16, 2023

(54) KETOGENIC FASTING-MIMICKING DIET TO IMPROVE KIDNEY FUNCTION

(71) Applicants: UNIVERSITY OF SOUTHERN CALIFORNIA, Los Angeles, CA (US); CHILDREN'S HOSPITAL LOS ANGELES, Los Angeles, CA (US)

(72) Inventors: Valter D. LONGO, Los Angeles, CA (US); Roberta BUONO, Los Angeles, CA (US); Laura PERIN, Los Angeles, CA (US); Valentina VILLANI, Los Angeles, CA (US)

(73) Assignees: UNIVERSITY OF SOUTHERN CALIFORNIA, Los Angeles, CA (US); CHILDREN'S HOSPITAL LOS ANGELES, Los Angeles, CA (US)

(21) Appl. No.: 17/794,497

(22) PCT Filed: Jan. 21, 2021
(86) PCT No.: PCT/US2021/014362

§ 371 Date: Jul. 21, 2022

Related U.S. Application Data
(60) Provisional application No. 62/963,652, filed on Jan. 21, 2020.

(51) Int. Cl. A23L 33/16; A23L 33/00; A23L 33/125

(52) U.S. Cl. CPC A23L 33/16; A23L 33/30; A23L 33/125; A23V 2002/00

(57) ABSTRACT

A dietary composition and method for treating kidney damage and/or disease with a fasting-mimicking diet is provided. The method includes a step of identifying a subject having kidney damage. A low sodium fasting-mimicking diet with a specific dietary composition is administered to the subject for a first time period of 1 to 10 days.

### CROSS-REFERENCE TO RELATED APPLICATIONS

This application claims the benefit of U.S. Provisional Application Serial No. 62963652 filed Jan. 21, 2020, the disclosure of which is hereby incorporated in its entirety by reference herein.

### TECHNICAL FIELD

In at least one aspect, the present invention is related to dietary methods for treating kidney damage and/or disease resulting from injury by progressive diseases (of the glomerulus and tubules) such as diabetic nephropathy, membranous nephropathy, focal segmental glomerulosclerosis, crescentic glomerulonephritis, membranoproliferative glomerulonephritis, mesangial proliferative glomerulonephritis, intraductal proliferative glomerulonephritis, mesangial capillary glomerulonephritis, ischemic nephropathy, glomerular disease based on systematic diseases, vascular disease, metabolic diseases, hereditary renal lesions (such as polycystic kidney disease, Alport Syndrome, etc.) or transplanted glomerular lesions. In addition, the disease can be a result of acute and non-acute (including trauma and chemical overdose).

### BACKGROUND

A major hallmark of end-stage renal disease (ESDR) is the irreversible loss of the glomerular filtration barrier. The only treatment options are dialysis or transplantation. To date, no cures are available to restore kidney function in ESDR patients¹. While dietary recommendations are currently given to patients with kidney disease to minimize the burden of the disease itself and slow down the progression, no diet has proven effective in restoring renal function.

Intermittent or Periodic Fasting has been reported to promote lifespan extension and provide a range of protective effects, including stem cell generation in different systems and protection against chemotherapy side effects or cancer progression²⁻⁶.

Accordingly, there is a need for improved methods for treating renal disease.

### SUMMARY

In at least one aspect, the present invention provides compositions and methods for a new ketogenic fasting-mimicking diet having low sodium.

In another aspect, the present invention provides a method and diet that induces the activation of circulating mesenchymal stem cells, which can promote the regeneration of damaged kidney tissues. These results strongly support a role for FMD diets in the stimulation of regeneration, repair, and restoration of kidney function.

In another aspect, a method for treating kidney damage is provided. The method includes a step of identifying a subject having glomerulosclerosis. A low sodium fasting-mimicking diet (a low sodium FMD) is administered to the subject for a first time period of 1 to 10 days. Characteristically, the low sodium FMD provides less than 1000 mg of sodium chloride per day of the low sodium FMD. The low sodium FMD includes kilocalories from carbohydrates in an amount from about 30 to 70 percent of the total kilocalories of the low sodium FMD, kilocalories from protein in an amount less than about 10 percent of the total kilocalories of the low sodium FMD, and kilocalories from fat carbohydrates in an amount from about 30 to 70 percent of the total kilocalories of the low sodium FMD.

In another aspect, a method for treating glomerulosclerosis is provided. The method includes a step of identifying a subject having glomerulosclerosis. A low sodium fasting-mimicking diet (a low sodium FMD) is administered to the subject for a first time period of 1 to 10 days. Characteristically, the low sodium FMD provides less than 1000 mg of sodium chloride per day of the low sodium FMD. The low sodium FMD includes kilocalories from carbohydrates in an amount from about 30 to 70 percent of the total kilocalories of the low sodium FMD, kilocalories from protein in an amount less than about 10 percent of the total kilocalories of the low sodium FMD, and kilocalories from fat carbohydrates in an amount from about 30 to 70 percent of the total kilocalories of the low sodium FMD.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1: Flow cytometry analyses of blood to identify circulating mesenchymal stem cells (CD45-CD34-CD90+) during one FMD cycle after PAN damage. Data are shown as Mean±SEM, N=5. ** p<0.01

FIG. 2: Albumin/Creatinine ratio ACR analyses and BUN after six FMD cycles. Data are shown as Mean±SEM, N=10. *p<0.05, ** p<0.01. This is the graph for the BUN to add

FIG. 3: Evaluation of glomerular sclerosis after six FMD cycles. Data are shown as Mean±SEM, N=10. *p<0.05, ** p<0.01. This is the graph for glomerular sclerosis. Quantification of glomerulosclerosis index (G.I.). An average of 38 glomeruli per rat were analyzed, with n=3-5 rats/group (****:p<0.0001). Rats injected with PAN and fed ad libitum for the duration of the experiment had a significantly increased G.I. of 3 compared to W.T. rats. Rats who received 6 cycles of either FMD or LS-FMD following PAN injection had a G.I. comparable to healthy W.T. rats and significanlty lower than the PAN group (**:p<0.001*).

FIG. 4: Evaluation of EGFR (epidermal growth factor receptor) in the urine after six FMD cycles. Data are shown as Mean±SEM, N=10. *p<0.05, **p<0.01. We observe an increase_of EGFR (soluble receptor) in the urine of PAN rats compared to W.T. animals. We also observe levels comparable to healthy rats in both FMD diet groups (significantly reduced compared to PAN). (**:p<0.001*).

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4.... 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numeral quantity, in a refinement, the term “less than” includes a lower non-included limit that is 5 percent of the number indicated after “less than” truncated or rounded to 2 significant figures. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, or 1 percent of the number indicated after “less than” truncated or rounded to 2 significant figures.

Unless stated to the contrary, weight percentages for an ingredient of a fasting-mimicking diet package component are referenced to the total weight of that component.

Diet compositions described for a fifth day of the FMD and any packages thereof can also be provided for any additional days if the FMD is extended beyond 5 days.

In numerical ranges having 0 as a lower limit, a refinement can have an alternative lower limit that is 5 percent of the upper limit truncated or rounded to 2 significant figures. For example, “0 to 20” includes a lower non-included limit of 1 in a refinement. Therefore, this refinement of “0 to 20” includes a range from 1 to 20. In another refinement, numerical ranges having 0 as a lower limit includes a lower limit that is, in increasing order of preference, 20 percent, 10 percent, 5 percent, or 1 percent of the upper limit truncated or rounded to 2 significant figures.

Throughout this application, where publications are referenced, the disclosures of these publications in their entireties are hereby incorporated by reference into this application to more fully describe the state of the art to which this invention pertains.

The term “subject” refers to a human or other animal, including birds and fish as well as all mammals such as primates (particularly higher primates), horses, birds, fish sheep, dogs, rodents, guinea pigs, pig, cat, rabbits, and cows.

Abbreviations:

“BUN” means blood urea nitrogen.

“EGFR” mated glomerular filtration rate.

“ESDR” means end stage renal disease.

“FMD” is fasting-mimicking diet.

“kcal” means kilocalorie.

“LS-FMD” means low sodium FMD.

“L.S.” means low sodium.

“MRI” means magnetic resonance imaging.

“CAT” means computerized axial tomography.

“IVP” means intravenous pyelogram.

The terms “kilocalorie” (kcal) and “Calorie” refer to the food calorie.

The term “subject” refers to a human or animal, including all mammals such as primates (particularly higher primates), sheep, dog, rodents (e g., mouse or rat), guinea pig, goat, pig, cat, rabbit, and cow.

The term “fasting-mimicking diet” (FMD) means a diet that mimics the effects of fasting typically by providing a subject with at most 50% of their normal caloric intake but with some nutritional component so that fasting is mimicked while a subject is nourished and not completely starved. Examples of useful fasting-mimicking and enhancing diets and methods for monitoring the effects of these diets on markers such as IGF-1 and IGFBP1 in the context of the present invention are set forth in U.S. Pat. Application Serial. No. 14/273,946 filed May 9, 2014; Serial. No. 14/497,752 filed Sep. 26, 2014; Serial. No. 12/910,508 filed Oct. 22, 2010; Serial. No. 13/643,673 filed Oct. 26, 2012; Serial. No. 13/982,307 filed Jul. 29, 2013; Serial. No. 14/060,494 filed Oct. 22, 2013; Serial. No. 14/178,953 filed Feb. 12, 2014; Serial. No. 14/320,996 filed Jul. 1, 2014; Serial. No. 14/671,622 filed Mar. 27, 2015; the entire disclosure of these patent applications is hereby incorporated by reference. The fasting-mimicking diet set forth in U.S. Pat. Application Serial. Nos. 14/060,494 and 14/178,953 are found to be particularly useful in the present invention. Additional examples of FMD diets are found in U.S. Pat. Application Serial. No. 15/148,251 and WIPO Pub. No. WO2011/050302 and WIPO Pub. No. WO2011/050302; the entire disclosures of which are hereby incorporated by reference. In a refinement, the low sodium FMD used herein can be constructed by limiting to amount of sodium (i.e., sodium chloride) in these prior FMDs. In a refinement, the FMD is a ketogenic FMD. Similarly, the low sodium FMD can be a low sodium ketogenic FMD. Therefore, in the descriptions set forth herein, the term ketogenic FMD can be replaced by FMD and vice versa as needed. Similarly, in the descriptions set forth herein the term ketogenic fasting-mimicking diet can be replaced by fasting-mimicking diet and vice versa as needed.

In an embodiment, a method for treating kidney damage, and in particular, end ESDR is provided. The method includes a step of identifying a subject having kidney damage and/or kidney disease, including but not limited to ESDR and/or disease resulting from injury of progressive disease (of the glomerulus and tubules) such as diabetic nephropathy, membranous nephropathy, focal segmental glomerulosclerosis, crescentic glomerulonephritis, membranoproliferative glomerulonephritis, mesangial proliferative glomerulonephritis, intraductal proliferative glomerulonephritis, mesangial capillary glomerulonephritis, ischemic nephropathy, glomerular disease based on systematic diseases, vascular disease, metabolic diseases, hereditary renal lesions (such as polycystic kidney disease, Alport Syndrome, etc.) or transplanted glomerular lesions. In addition, the disease can be a result of acute and non-acute (including trauma and chemical overdose). Methods for identifying kidney damage include, but are not limited to, blood tests (e.g., glomerular filtration rate), urine tests, imaging studies (e.g., MRI, CAT scans, IVP, and the like), and kidney biopsies. The subject is then administered a low sodium FMD (LS-FMD) for a first time period. In a refinement, the first time period is 1 to 10 days (i.e., 1, 2, 3, 4, 5, 6, 7, 8, 9, or 10 days). Typically, the first time period is 5 or 6 days. In a further variation, the low sodium FMD is repeating at predefined intervals. Such intervals can be from 5 to 26 days or longer. The total duration for treatment (i.e., the treatment time period) can be from a month to several years (e.g., 1, 2, 3, 4, or 5 years) during with the low sodium FMD is periodically or repeatedly administered. During and/or after treatment the subject’s kidney disease can be re-evaluated using the methods for identifying kidney damage as set forth above. It should be appreciated that although low sodium ketogenic FMD diets are optimal for treating kidney disease, ketogenic FMD’s that are not low sodium can also provide a benefit for treating kidney disease. Therefore, a normal ketogenic FMD (i.e., not low sodium) can be substituted in the descriptions of the low sodium FMD and vice versa as needed.

Advantageously, the low sodium ketogenic FMD decreases proteinuria and BUN and ameliorates glomerular histology and stabilizes EGFR. The low sodium FMD re-establishes normal value of EGFR in urine. Average estimated GFR in different age groups is 116 mL/min/1.73 m² for ages 20-29 years, 107 mL/min/1.73 m² for ages 30-39 years, 99 mL/min/1.73 m for ages 40-49 years, 93 mL/min/1.73 m for ages 50-59 year, 85 mL/min/1.73 m for ages 60-69 year, and 75 mL/min/1.73 m for ages 70 years or older. In a refinement, cycles of the low sodium FMD are repeated a plurality of times until the EGFR are within a predetermined percent (e.g., 50%, 40%, 30%, or 20%) of the age appropriate normal value for the subject being treated. Normal ranges for BUN are Adults up to 60 years of age: 6-20 mg/dL for adults 18 to 60 years of age and: 8-23 mg/dL for adults over 60 years of age. In a refinement, cycles of the low sodium ketogenic FMD are repeated a plurality of times until the BUN are within a predetermined percent (e.g., 50%, 40%, 30%, or 20%) of the age appropriate upper or lower normal ranges for the subject being treated. In another refinement, cycles of the low sodium ketogenic FMD are repeated a plurality of times until the albumin concentrations in a subject’s urine are less than a predetermined amount (e.g., less than, in increasing order of preference, 300 mg/dL, 100 mg/dL, or 30 mg/dL.) The amount of albumin in urine can be determined by Microalbumin Test or by the protein dipstick test.

In some variations, a refeeding diet is administered to the subject for a second time period. The refeeding diet provides an overall calorie consumption that is within 10 percent of a subject’s normal calorie consumption. In this context, normal calorie consumption is the number of kilocalories needed to maintain the subject’s weight at their pretreatment weight (e.g., the day before treatment is commenced) or to maintain a predetermined target weight. Therefore, the refeeding diet can be referred to as a “refeeding diet.” Although the present invention is not significantly limited by the second time period, the second time period can be from 7 days to 6 months or longer. Typically, the second time period is from 5 days to 26 days or longer following the low sodium diet. In some refinements, the refeeding diet provides at most, in increasing order of preference, 2500 kcal/day, 2400 kcal/day, 2300 kcal/day, 2200 kcal/day, 2100 kcal/day, 2000 kcal/day, 1900 kcal/day, 1800 kcal/day, 1700 kcal/day, 1600 kcal/day, or 1500 kcal/day. In some further refinements, the refeeding diet provides at least, in increasing order of preference, 1200 kcal/day, 1300 kcal/day, 1400 kcal/day, 1500 kcal/day, 1600 kcal/day, 1700 kcal/day, or 1800 kcal/day. In a refinement, the low sodium ketogenic FMD is administered biweekly (i.e., every two weeks) after kidney damage has been identified. Advantageously, this new low sodium FMD increases circulating mesenchymal stem cell and improve kidney function.

Typically, the low sodium ketogenic FMD is based on human foods that allow nourishment during periods of low-calorie consumption and high fat. In a variation, the low sodium ketogenic FMD provides less than about 1000 mg per day of sodium. In a variation, the low sodium ketogenic FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day. In a refinement, the low sodium ketogenic FMD provides sodium (e.g., sodium chloride) in an amount that greater than or equal to, in increasing order of preference, 200 mg per day, 100 mg per day, 50 mg per day, 10 mg per day, , or 0 mg per day. The low sodium ketogenic FMD typically includes kilocalories from carbohydrates in an amount from about 30 to 70 percent of the total kilocalories of the FMD, kilocalories from protein in an amount less than about 10 percent of the total kilocalories of the FMD, and kilocalories from fat carbohydrates in an amount from about 30 to 70 percent of the total kilocalories of the FMD. In a refinement, the low sodium ketogenic FMD typically includes kilocalories from carbohydrates in an amount from about 35 to 50 percent of the total kilocalories of the FMD, kilocalories from protein in an amount from about 0.5 to 10 percent of the total kilocalories of the FMD, and kilocalories from fat carbohydrates in an amount from about 40 to 60 percent of the total kilocalories of the FMD. In a further refinement, the source of kilocalories is 42% from carbohydrates, 2% protein, and 47% fat. In some refinements, the diet is mainly composed of olive oil and vegetable soups.

In a variation, the low sodium ketogenic FMD diet provides less than 30 g of sugar on day 1 and less than 20 g of sugar on days 2-5 and any additional days of the diet. The diet should contain less than 28 g of proteins on day 1 and less than 18 g of proteins on days 2-5 and any additional days (e.g., days 6-10) of the diet. , mostly or completely from plant-based sources. The diet should contain between 20 and 30 grams of monounsaturated fats on day 1 and 10-15 grams of monounsaturated fats on days 2-5 and any additional days (e.g., days 6-10) of the diet. The diet should contain between 6 and 10 grams of polyunsaturated fats on day 1 and 3-5 grams of polyunsaturated fats on days 2-5 and any additional days (e.g., days 6-10) of the diet. The diet should contain less than 12 g of saturated fats on day 1 and less than 6 grams of saturated fats on days 2-5 and any additional days (e.g., days 6-10) of the diet. Typically, the fats on all days are derived from a combination of the following: Almonds, Macadamia Nuts, Pecans, Coconut, Coconut oil, Olive Oil, and Flaxseed. In a refinement, the FMD diet includes over 50% of the recommended daily value of dietary fiber on all days. In a further refinement, the amount of dietary fiber is greater than 15 grams per day on all five days. The diet should contain 12-25 grams of glycerol per day on days 1-5 (or 2-5) and any additional days (e.g., days 6-10) of the diet. In a refinement, glycerol is provided at 0.1 grams per pound body weight/day. Characteristically, each of the components of the low sodium ketogenic FMD are chosen to contain or prepared with components having low amounts of sodium (i.e., sodium chloride). For some components (e.g., vegetables), the components can be washed with water (to extract the sodium) such that the sodium chloride is removed.

In a variation, the low sodium ketogenic FMD includes the following micronutrients (at least 95% non-animal based): over 5,000 IU of vitamin A per day (days 1-5 and any additional days); 60-240 mg of vitamin C per day (days 1-5); 400-800 mg of calcium per day (days 1-5 and any additional days); 7.2-14.4 mg of iron per day (days 1-5 and any additional days); 200-400 mg of magnesium per day (days 1-5 and any additional days); 1-2 mg of copper per day (days 1-5 and any additional days); 1-2 mg of manganese per day (days 1-5 and any additional days); 3.5-7 mcg of selenium per day (days 1-5 and any additional days); 2-4 mg of Vitamin B1 per day (days 1-5 and any additional days); 2-4 mg of Vitamin B2 per day (days 1-5 and any additional days); 20-30 mg of Vitamin B3 per day (days 1-5 and any additional days); 1-1.5 mg of Vitamin B5 per day (days 1-5 and any additional days); 2-4 mg of Vitamin B6 per day (days 1-5 and any additional days); 240-480 mcg of Vitamin B9 per day (days 1-5 and any additional days); 600-1000 IU of Vitamin D per day (days 1-5 and any additional days); 14-30 mg of Vitamin E per day (days 1-5 and any additional days); over 80 mcg of Vitamin K per day (days 1-5 and any additional days); 16-25 mcg Vitamin B12 are provided during the entire low sodium ketogenic FMD; and docosahexaenoic acid (DHA, algae-derived) provided over the first five day and/or any additional days. For example, 600 mg can be given over the first 5 days of the FMD. The FMD diet provides high micronutrient content mostly (i.e., greater than 50 percent by weight) from natural sources such as kale, cashews, yellow bell pepper, onion, lemon juice, yeast, turmeric, mushroom, carrot, olive oil, beet juice, spinach, tomato, collard, nettle, thyme, salt, pepper, Vitamin B12 (Cyanocobalamin), beets, butternut squash, oregano, tomato juice, orange juice, celery, romaine lettuce, cumin, orange rind, citric acid, nutmeg, cloves, and combinations thereof. Table 1 provides an example of additional micronutrient supplementation that can be provided in the FMD diet:

Table 1

Micronutrient Supplementation

| | Supplement | Formula | Amount | Amount Range | Unit |
|---|---|---|---|---|---|
| Vit A | | | 1250 IU | 900-1600 | IU |
| Vit C | Ascorbic Acid | C₆H₈O₆ | 15.0000 | 10-20 | mg |
| Ca | Calcium Carbonate | CaCO₃ | 80.0000 | 60-100 | mg |
| Fe | Ferrous Fumarate | C₄H₂FeO₄ | 4.5000 | 3-6 | mg |
| Vit D3 | Cholecalciferol | C₂₇H₄₄O | 0.0025 | 0.001-0.005 | mg |
| Vit E | d1-Alpha Tocopheryl Acetate | C₂₉H₅₀O₂ | 5.0000 | 3-7 | mg |
| Vit K | Phytonadione | | 0.0200 | 0.1-0.04 | mg |
| Vit B1 | Thiamine Mononitrate | C₁₂H₁₇N₅O₄S | 0.3750 | 0.15-0.5 | mg |
| Vit B2 | Riboflavin E101 | C₁₇H₂₀N₄O₆ | 0.4250 | 0.2-0.6 | mg |
| Vit B3 | Niacinamide | C₆H₆N₂O | 5.000 | 3-7 | mg |
| Vit B5 | Calcium Pantothenate | C₁₈H₃₂CaN₂O₁₀ | 2.5000 | 1.5-4.0 | mg |
| Vit B6 | Pyridoxine Hydrochloride | C₈H₁₁NO₃ · HCl | 0.5000 | 0.3-0.7 | mg |
| Vit B7 | Biotin | C₁₀H₁₆N₂O₃S | ^{}0.0150 | 0.01-0.02 | mg |
| Vit B9 | Folic Acid | C₁₉H₁₉N₇O₆ | 0.1000 | 0.07-0.14 | mg |
| Vit B12 | Cyanocobalamin | C₆₃H₈₈CoN₁₄O₁₄P | 0.0015 | 0.001-0.002 | mg |

| | | | | | |
|---|---|---|---|---|---|
| Cr | Chromium Picolinate | Cr(C6H4NO2)3 | 0.0174 | 0.014-0.022 | mg |
| Cu | Cupric Sulfate | CuS04 | 0.2500 | 0.18-0.32 | mg |
| I | Potassium Iodide | KI | 0.0375 | 0.03-0.045 | mg |
| Mg | Magnesium Oxide | MgO | 26.0000 | 20-32 | mg |
| Mn | Manganese Sulfate | MnSO₄ | 0.5000 | 0.3-0.7 | mg |
| Mo | Sodium Molybdate | Na₂MoO₄ | 0.0188 | 0.014-0.023 | mg |
| Se | Sodium Selenate | Na₂O₄Se | 0.0175 | 0.014-0.023 | mg |
| Zn | Zinc Oxide | ZnO | 3.7500 | 3-5 | mg |

In another embodiment, a diet package for implementing the low sodium ketogenic fasting-mimicking diets set forth above is provided. In general, the diet package includes rations for implementing the fasting-mimicking diets set forth above. The sources of these rations are also set forth above. In particular, the rations are divided into portions for each cycle of the low sodium ketogenic fasting-mimicking diet. In a variation, the diet package includes rations having (i.e., providing) kilocalories from carbohydrates in an amount from about 30 to 70 percent of the total kilocalories of the FMD, kilocalories from protein in an amount less than about 10 percent (e.g., 0.1 to 10 percent protein of the total kilocalories of the FMD, and kilocalories from fat carbohydrates in an amount from about 30 to 70 percent of the total kilocalories of the FMD. In a refinement, the diet package includes rations having kilocalories from carbohydrates in an amount from about 35 to 50 percent of the total kilocalories of the FMD, kilocalories from protein in an amount from about 0.5 to 10 percent of the total kilocalories of the FMD, and kilocalories from fat carbohydrates in an amount from about 40 to 60 percent of the total kilocalories of the FMD. In a further refinement, the diet package includes rations having kilocalories is 42% from carbohydrates, 2% protein, and 47% fat. In some refinements, the diet is mainly composed of olive oil and vegetable soups. In a refinement, the rations are prepared such that the low sodium ketogenic FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day.

Alternatively, the rations are divided into portions for each day of the fasting-mimicking diet. In another variation, the rations are divided into portions for each meal of the low sodium ketogenic fasting-mimicking diet. For example, the diet package includes a first set of rations for a low sodium ketogenic fasting-mimicking diet to be administered for the first time period to a subject, the low sodium ketogenic fasting-mimicking diet providing from 4.5 to 7 kilocalories per pound of subject for a first day and 3 to 5 kilocalories per pound of subject per day for a second to fifth day (and any additional days) of the low sodium ketogenic FMD. In a refinement, the low sodium ketogenic FMD is a low protein diet. In a variation, the diet package includes rations that provide less than 30 g of sugar on the first day; less than 20 g of sugar on the second to fifth days; less than 28 g of proteins on the first day; less than 18 g of proteins on the second to fifth days; 20 to 30 grams of monounsaturated fats on the first day; 10 to 15 grams of monounsaturated fats on the second to fifth days; between 6 and 10 grams of polyunsaturated fats on the first day; 3 to 5 grams of polyunsaturated fats on the second to fifth days; less than 12 g of saturated fats on the first day; less than 6 grams of saturated fats on the second to fifth days; and 12 to 25 grams of glycerol per day on the second to fifth days. In a refinement, the diet package further includes sufficient rations to provide the micronutrients set forth above. In a further refinement, the diet package provides instructions providing details of the methods set forth above. In a refinement, the rations are prepared such that the low sodium ketogenic FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day.

In a variation, an FMD and diet package thereof that can be used to treat kidney disease as set forth above is provided in U.S. Pat. Publication 20180228198, filed Feb. 14, 2017; the entire disclosure of which is hereby incorporated by reference. In a variation, this FMD is formed with reduced or no sodium added to form a low sodium ketogenic FMD. In some refinements, the amounts of salt and/or the ranges of salt are increasing order of preference, 50%, 40%, 30%, 20%, 10%, 5%, or 1% of the amounts listed in U.S. Pat. Publication 20180228198.

In a refinement, the low sodium ketogenic fasting-mimicking diet package provides daily meal portions for a predetermined number of days to treat kidney disease as set forth above. Typically, the predetermined number of days is from 1 to 10 days (i.e., 1, 2, 3, 4, 5, 6, 7, 8, 9, or 10 days). In a particularly useful variation, the predetermined number of days is 5 or 6 days. In some variations, the low sodium ketogenic fasting-mimicking diets set forth herein provide a subject at most, in increasing order of preference, 75%, 50%, 40%, 30%, or10% of the subject’s normal caloric intake or the daily recommended caloric intake for a subject. In a refinement, the low sodium ketogenic fasting-mimicking diet provides at least, in increasing order of preference, 5%, 10%, or 20% of the subject’s normal caloric intake or the daily recommended caloric intake for a subject. The subject’s normal caloric intake is the number of kcal that the subject consumes to maintain his/her weight. The subject’s normal caloric intake may be estimated by interviewing the subject or by consideration of a subject’s weight. As a rough guide, the subject’s normal caloric intake is on average 2600 kcal/day for men and 1850 kcal/day for women. In certain instances, the low sodium ketogenic fasting-mimicking diet provides the subject with 700 to 1200 kcal/day. In a particularly useful refinement, the low sodium ketogenic fasting-mimicking diet provides a male subject of average weight with about 1100 kcal/day and a female subject of average weight with 900 kcal/day. In some variations, the diet from the diet package is administered on consecutive days. In another variation, the daily meal portions are provided for only one day a week for at least a month.

In a variation as set forth in U.S. Pat. Publication 20180228198, a fasting-mimicking diet package includes a kale cracker composition, a first vegetable broth composition, a mushroom soup composition, a tomato soup composition, a quinoa-containing minestrone soup composition, a bean-containing minestrone soup composition, and a pumpkin soup composition. Characteristically, the daily meal portions are packaged into meal servings or into a total daily serving to be divided into meals. In a refinement, the fasting-mimicking diet package further includes a nut-containing nutrition bar, a cocoa-containing nutrition bar, a first olive-containing composition, a first vegetable broth composition, a tea composition that includes spearmint, a energy drink composition, a micronutritional composition, and an algal oil composition. In a further refinement, the fasting-mimicking diet package further includes a second olive-containing composition, a second vegetable broth composition, a tea composition that includes spearmint and lemon, and a tea composition that includes hibiscus. In a refinement, the amounts of salt and/or the ranges of salt in each of these components are increasing order of preference, 50%, 40%, 30%,20%, 10%, 5%, or 1% of the amounts listed in U.S. Pat. Publication 20180228198.

In a variation of the embodiments set forth above, the fasting-mimicking diet package includes daily meal portions that provide less than 40 grams of sugar for day 1, less than 30 grams of sugar for days 2 to 5 and any remaining days, less than 28 grams of protein for day 1, less than 18 grams of protein for days 2 to 5 and any remaining days, 20-30 grams of monounsaturated fats or more to reach the desired caloric intake (i.e., a predetermined caloric intake) for day 1, 6-10 grams of polyunsaturated fats or more to reach the desired caloric intake for day 1, 2-12 grams of saturated fats or more to reach the desired caloric intake for day 1, 10-15 grams of monounsaturated fats or more to reach the desired caloric intake for days 2 to 5 and any remaining days, 3-5 grams of polyunsaturated fats or more to reach the desired caloric intake for days 2 to 5 and any remaining days, 1-6 grams of saturated fats or more to reach the desired caloric intake for days 2 to 5, or any remaining days, and a micronutrient composition on each day and any remaining days.

In another variation of the embodiments set forth above, the fasting-mimicking diet package includes daily meal portions 8-10 kcal per kilogram body weight for each diet day. In this variation, the fasting-mimicking diet provides less than 30 grams of sugar for each diet day, less than 18 grams of protein for each diet day, 9-15 grams of monounsaturated fats or more to reach the desired caloric intake for each diet day, and 2.5-4.5 grams of polyunsaturated fats or more to reach the desired caloric intake for each diet day and 1-5.5 grams of saturated fats or more to reach the desired caloric intake for each diet day. Higher levels of the fats listed above can be provided for higher FMD formulation providing up to 100% of the normal caloric intake to subjects.

In still another variation of the embodiments set forth above, the fasting-mimicking diet package includes daily meal portions that provide 5-8 kcal per kilogram body weight for each diet day. In this variation, the fasting-mimicking diet provides less than 20 grams of sugar for each diet day, less than 12 grams of protein for each diet day, and 6.5-10 grams of monounsaturated fats or more to reach the desired caloric intake for each diet day, 2.5-4.5 grams of polyunsaturated fats or more to reach the desired caloric intake for each diet day and 1.5-4 grams of saturated fats or more to reach the desired caloric intake for each diet day.

In still another variation of the embodiments set forth above, the fasting-mimicking diet package includes daily meal servings that provide 0-3 kcal per kilogram body weight for each diet day. In this variation, the fasting-mimicking diet provides less than 5 grams of sugar for each diet day, less than 3 grams of protein for each diet day, and less than 2.5 grams of monounsaturated fats for each diet day, less than 1 grams of polyunsaturated fats for each diet day and less than 1 grams of saturated fats for each diet day.

In an embodiment, the nutritional requirements for the fasting-mimicking diet set forth above can be realized by a diet package with certain specific meal components. In one variation, the fasting-mimicking diet package 10 provides daily meal portions for a predetermined number of days are set forth above (e.g., 1, 2, 3, 4, 5, 6, 7, 8, 9, or 10 days). The fasting-mimicking diet package 10 includes a kale cracker composition, a first vegetable broth composition, a mushroom soup composition, a tomato soup composition, a quinoa-containing minestrone soup composition, a bean-containing minestrone soup composition, and a pumpkin soup composition. Characteristically, the daily meal portions are packaged into meal servings or into a total daily serving to be divided into meals. In a refinement, the fasting-mimicking diet package further includes a nut-containing nutrition bar, a cocoa-containing nutrition bar, a first olive-containing composition, a first vegetable broth composition (item 32), a tea composition that includes spearmint, a energy drink composition, a micronutritional composition, and an algal oil composition. In a further refinement, the fasting-mimicking diet package further includes a second olive-containing composition, a second vegetable broth composition, a tea composition that includes spearmint and lemon, and a tea composition that includes hibiscus. It should be appreciated that each of the soup, broth, tea, and energy compositions set forth herein are designed to have added water when consumed.

In another variation of a fasting-mimicking diet package, diet package 10 includes a nut-containing nutrition bar, a cocoa-containing nutrition bar, a first olive-containing composition (item 30), a kale cracker composition, a vegetable soup composition, a first vegetable broth composition, a tea composition that includes spearmint, an energy drink composition (item 36), a micronutritional composition, and an algal oil composition. Characteristically, the daily meal portions are packaged into meal servings or into a total daily serving to be divided into meals. This diet package also includes daily meal portions for a predetermined number of days as set forth above, with the daily meal portions being packaged into meal servings or into a total daily serving to be divided into meals. In a refinement, the fasting-mimicking diet package further includes a mushroom soup composition, a tomato soup composition, a quinoa-containing minestrone soup composition, and a pumpkin soup composition. In a further refinement, the fasting-mimicking diet package further includes a second olive-containing composition, a second vegetable broth composition, a bean-containing minestrone soup composition, a tea composition that includes spearmint and lemon, and a tea composition that includes hibiscus.

As set forth above, the fasting-mimicking diet packages include specific meal components. Typically, compositions are as follows. The nut-containing nutrition bar includes almond meal and macadamia nuts. The cocoa-containing nutrition bar includes almond butter, almonds, and brown rice crispy (e.g., brown puffed rice). The mushroom soup composition includes brown rice powder, carrots, inulin, and mushrooms. The bean-containing minestrone soup composition includes white beans, cabbage, and potatoes. The first vegetable broth composition includes carrots, maltodextrin, celery, spinach, and tomatoes. The second vegetable broth composition includes carrots, maltodextrin, celery, spinach, soy lecithin, and tomatoes. The energy drink composition includes glycerin and water. The algal oil composition includes schizocatrium algae oil. The micronutrient composition includes beet root powder, calcium carbonate, carrots, collard leaf, kale leaf, and tomatoes. In a refinement, the micronutrient composition includes Vit A, Vit C, Ca, Fe, Vit D3, Vit E, Vit K, Vit B1, Vit B2, Vit B3, Vit B5, Vit B6, Vit B7, Vit B9, Vit B12, Cr, Cu, I, Mg, Mn, Mo, Se, and Zn.

In a refinement, the nut-containing nutrition bar (L-Bar Nut based) includes almond meal and macadamia nuts. In a refinement, the nut-containing nutrition bar (L-Bar Nut based) includes almond meal preferably in an amount of 20 to 35 weight %; coconut preferably in an amount of 2 to 10 weight %; coconut oil preferably in an amount of 1 to 8 weight %; flax seed meal preferably in an amount of 1 to 8 weight %; honey preferably in an amount of 10 to 30 weight %; macadamia nuts preferably in an amount of 10 to 30 weight %; pecans preferably in an amount of 10 to 25 weight %; salt preferably in an amount of 0.0 to 0.8 weight % (e.g., 0.0.2 to 0.2 weight % or 0.1 to 0.8 weight %); and optionally vanilla preferably in an amount of 0.3 to 1.5 weight %.

In a refinement, the cocoa-containing nutrition bar (L-Bar ChocoCrisp) includes almond butter, almonds, and brown rice crispy (PGP10235). In a refinement, the cocoa-containing nutrition bar (L-Bar ChocoCrisp) includes almond butter preferably in an amount of 10 to 25 weight %; almonds preferably in an amount of 3 to 12 weight %; brown rice crispy (PGP10235) preferably in an amount of 10 to 25 weight %; brown rice syrup preferably in an amount of 2 to 8 weight %; chocolate liquor preferably in an amount of 1 to 4 weight %, cocoa butter preferably in an amount of 0.4 to 1.6 weight %; cocoa powder preferably in an amount of 4 to 12 weight %; fiber syrup SF75 preferably in an amount of 18 to 38 weight %, flaxseed oil preferably in an amount of 1 to 3 weight %; salt preferably in an amount of 0.0 to 0.4 weight % (0.1 to 0.4 weight % or 0.05 to 0.1 weight percent) and sugar preferably in an amount of 1 to 6 weight %.

In a refinement, the first olive-containing composition (sea salt version) includes olives, olive oil, and sea salt. In a refinement, the first olive-containing composition (sea salt) includes lactic acid preferably in an amount of 0.3 to 1 weight %; oil (olive) preferably in an amount of 2 to 6 weight %; olives (raw, green pitted) preferably in an amount of 50 to 97 weight %; salt (reg., kosher, sea salt) preferably in an amount of 0.0 to 3 weight % (e.g., 0.2 to 0.8 weight percent or 0.8 to 3 weight %) ; and thyme preferably in an amount of 0.1 to 0.5 weight %.

In a refinement, the second olive-containing composition (garlic version) includes olives, olive oil, and garlic. In a refinement, the second olive-containing composition (garlic) includes garlic preferably in an amount of 0.1 to 0.6 weight %; lactic acid preferably in an amount of 0.3 to 1 weight %; oil (olive) preferably in an amount of 2 to 6 weight %; olives (raw, green pitted) preferably in an amount of 50 to 97 weight %; salt (reg., kosher, sea salt) preferably in an amount of 0.0 to 3 weight % (e.g., 0.2 to 1 weight percent or 0.8 to 3 weight %); thyme preferably in an amount of 0.1 to 0.5 weight %.

In a refinement, the kale cracker composition includes kale, almonds, tapioca flour, and optionally sesame seeds. In another refinement, the kale cracker composition includes almonds preferably in an amount of 15 to 40 weight %; black pepper preferably in an amount of 0.1 to 0.4 weight %; chia seeds preferably in an amount of 3 to 10 weight %; chili pepper preferably in an amount of 0.4 to 1.2 weight %; cumin seeds preferably in an amount of 0.3 to 0.9 weight %; flax seeds preferably in an amount of 3 to 10 weight %; garlic preferably in an amount of 0.02 to 0.04 weight %; kale preferably in an amount of 2 to 6 weight %; oil (sun flower) preferably in an about of 2 to 7 weight %; onion (powder, minced) typically in an amount of 0.3 to 0.9 weight%; oregano preferably in an amount of 0.01 to 0.06 weight %; salt preferably in an amount of 0 to 4 weight % (e.g., 0.2 to 1 weight percent or 1 to 4 weight %); sesame seeds preferably in an amount of 15 to 35 weight %; sugar (coconut) preferably in an amount of 1 to 5 weight %; tapioca flour preferably in an amount of 10 to 30 weight %; vinegar (coconut) preferably in an amount of 1 to 4 weight %; water (purified) preferably in an amount of 2 to 12 weight %; and yeast extract preferably in an amount of 0.3 to 1 weight %.

In another refinement, the kale cracker composition includes kale, flax seeds golden, sesame seeds, and sunflower seeds. In another refinement, the kale cracker composition includes apple cider vinegar preferably in an amount 1 to 3 weight %; black pepper preferably in an amount of 0.4 to 1.3 weight %; cashews preferably in an amount of 4 to 13 weight %; dill weed preferably in an amount of 0.4 to 1.3 weight %; flax seeds golden preferably in an amount of 13 to 40 weight %; hemp seeds preferably in an amount of 0.7 to 2 weight %; kale preferably in an amount of 14 to 42 weight %; onion, white, dried, (powder, minced) preferably in an amount of 0.5 to 1.6 weight %; pumpkin seeds preferably in an amount of 0.7 to 2 weight %; salt (reg. , kosher, sea salt) preferably in an amount of 0.0 to 2 weight %; (e.g., 0.2 to 0.5 weight percent or 0.7 to 2 weight %); Sesame seeds preferably in an amount of 2 to 8 weight %; sunflower seeds preferably in an amount of 10 to 30 weight %; and yeast extract preferably in an amount of 1 to 5 weight %.

In a refinement, the vegetable soup composition includes onions, tomatoes, spinach, green tree extract, optionally rice flour, optionally brown rice powder, optionally carrots, and optionally inulin, leeks, In a refinement, the vegetable soup composition includes basil (whole leaf, dried) preferably in an amount of 0.3 to 0.9 weight %; brown rice powder (whole grain) preferably in an amount of 3 to 12 weight %; carrot (dehydrated, puffed, powder, pieces) preferably in an amount of 4 to 14 weight %; green tea extract preferably in an amount of 0.02 to 0.06 weight %; inulin preferably in an amount of 5 to 15 weight %; leeks (granules -10+40) preferably in an amount of 1 to 5 weight %; oil (olive) preferably in an amount of 1 to 6 weight %; onion (powder, minced) preferably in an amount of 4 to 15 weight %; parsley preferably in an amount of 0.3 to 0.8 weight %; red bell peppers preferably in an amount of 1 to 5 weight %; rice flour preferably in an amount of 18 to 50 weight %; salt preferably in an amount of 1 to 7 weight % (e.g., 2 to 7 weight % or 0.5 to 2 weight percent; spinach (leaf, powder) preferably in an amount of 0.4 to 1.5 weight %; tomatoes (fruit powder, sun dried, granules) preferably in an amount of 4 to 14 weight %; yeast extract preferably in an amount of 0.5 to 1.8 weight %. In the vegetable soup composition and any of the compositions set forth herein having rice flour, the rice flour can be glutinous or non-glutinous, milled or unmilled.

In another refinement, the vegetable soup composition includes carrots, inulin, leeks, onions and rice flour. In a refinement, the vegetable soup composition includes basil, whole leaf, dried preferably in an amount of 0.3 to 1 weight %; carrot (dehydrated, puffed, powder, pieces) preferably in an amount of 4 to 12 weight %; inulin preferably in an amount of 6 to 18 weight %; leeks in an amount of 1 to 5 weight %; oil (olive) preferably in an amount of 1 to 3 weight %; Onion, white, dried, (powder, minced) preferably in an amount of 10 to 30 weight %; parsley preferably in an amount of 0.3 to 1 weight %; potato preferably in an amount of 1 to 5 weight %; red pepper preferably in an amount of 1 to 6 weight %; rice flour in an amount of 13 to 40 weight %; salt (reg. , kosher, sea salt) in an amount of 0 to 12 weight % (e.g., 1 to 3 weight percent or 4 to 12 weight %); spinach (leaf, powder) preferably in an amount of 0.2 to 1 weight %; and tomatoes, (fruit powder, sun dried granules) preferably in an amount of 3 to 13 weight %.

In a refinement, the mushroom soup composition includes mushrooms, green tea extract, optionally brown rice powder, optionally carrots, and optionally inulin. In a refinement, the mushroom soup composition includes brown rice powder (whole grain) preferably in an amount of 10 to 30 weight %; carrot (dehydrated, puffed, powder, pieces) preferably in an amount of 3 to 12 weight %; green tea extract preferably in an amount of 0.02 to 0.06 weight %; inulin preferably in an amount of 3 to 12 weight %; mushrooms (European mix, powder, pieces) preferably in an amount of 6 to 18 weight %; oil (olive) preferably in an amount of 1 to 6 weight %; onion preferably in an amount of powder, minced) preferably in an amount of 3 to 12 weight %; parsley preferably in an amount of 0.1 to 0.5 weight %; rice flour preferably in an amount of 18 to 50 weight %; salt preferably in an amount of 2 to 8 weight % (e.g., 2 to 8 weight % or 0.5 to 2 weight percent); yeast extract preferably in an amount of 0.5 to 1.5 weight %.

In another refinement, the mushroom soup composition includes carrots, inulin, mushrooms, onions, and rice flour. In another refinement, the mushroom soup composition includes carrot (dehydrated, puffed, powder, pieces) preferably in an amount of 7 to 22 weight %; inulin preferably in an amount of 7 to 22 weight %; mushrooms (European mix), (powder & pieces)dehydrated preferably in an amount of 7 to 22 weight %; oil (olive) preferably in an amount of 0.6 to 2 weight %; Onion, white, dried, (powder, minced) preferably in an amount of 7 to 22 weight %; parsley preferably in an amount of 0.3 to 0.9 weight %; potato preferably in an amount of 0.6 to 2 weight %; rice flour preferably in an amount of 15 to 45 weight %; salt (reg. , kosher, sea salt) preferably in an amount of 0 to 18 weight % (e.g., 1 to 4 weight percent or 6 to 18 weight %); and yeast extract preferably in an amount of 0.7 to 2.2 weight %.

In a refinement, the tomato soup composition includes tomatoes, green tea extract, optionally inulin, and optionally onions. In a refinement, the tomato soup composition (new) includes basil (whole leaf, dried) preferably in an amount of 0.2 to 0.7 weight %; brown rice powder (whole grain) preferably in an amount of 1 to 5 weight %; green tea extract preferably in an amount of 0.02 to 0.06 weight %; inulin preferably in an amount of 7 to 20 weight %; oil (olive) preferably in an amount of 3 to 9 weight %; onion preferably (powder, minced) preferably in an amount of 4 to 12 weight %; parsley preferably in an amount of 0.1 to 0.6 weight %; rice flour preferably in an amount of 18 to 50 weight %; salt preferably in an amount of 2 to 9 weight % (e.g., 0.5 to 2 weight % or 2 to 9 weight %); tomatoes (fruit powder, sun dried, granules) preferably in an amount of 12 to 36 weight %; and yeast extract preferably in an amount of 0.5 to 3 weight %.

In another refinement, the tomato soup composition includes tomatoes, inulin, olives, onions, potatoes, and rice flour. In still another refinement, the tomato soup composition includes basil, whole leaf, dried preferably in an amount of 0.3 to 1 weight %; inulin preferably in an amount of 6 to 18 weight %; oil (olive) preferably in an amount of 4 to 14 weight %; onion, white, dried, (powder, minced) preferably in an amount of 8 to 24 weight %; parsley preferably in an amount of 0.3 to 0.9 weight %; potato preferably in an amount of 6 to 18 weight %; rice flour preferably in an amount of 9 to 27 weight %; salt (reg. , kosher, sea salt) preferably in an amount of 4 to 14 weight % (e.g., 1 to 3 weight % or 4 to 14 weight %); tomatoes, (fruit powder, sun dried granules) preferably in an amount of 8 to 24 weight %; and yeast extract preferably in an amount of 0.7 to 2.2 weight %.

In a refinement, the quinoa-containing minestrone soup composition includes quinoa, green tea extract, optionally olive oil, optionally cabbage, optionally potatoes, optionally rice flour, and optionally tomatoes and optionally no tumeric. In a refinement, the quinoa-containing minestrone soup composition includes basil (whole leaf, dried preferably in an amount of 0.7 to 2 weight %; broccoli powder preferably in an amount of 0.6 to 2 weight %; cabbage white (flakes) preferably in an amount of 3 to 10 weight %; carrot (dehydrated, puffed, powder, pieces) preferably in an amount of 3 to 10 weight %; celery preferably in an amount of 1 to 4 weight %; celery seeds (powder) preferably in an amount of 0.07 to 0.2 weight %; garlic preferably in an amount of 0.7 to 2 weight %; green tea extract preferably in an amount of 0.02 to 0.06 weight %; inulin preferably in an amount of 1 to 5 weight % ; leeks (granules -10+40), preferably in an amount of 0.7 to 2 weight %; oil (olive) preferably in an amount of 0.6 to 2 weight %; onion (powder, minced) preferably in an amount of 2 to 8 weight %; peas preferably in an amount of 3 to 10 weight %; potato preferably in an amount of 7 to 20 weight %; quinoa preferably in an amount of 7 to 20 weight %; rice flour preferably in an amount of 7 to 20 weight %; salt, preferably in an amount of 1 to 6 weight % (e.g., 0.2 to 1.5 weight % or 1 to 6 weight %); spinach (leaf, powder) preferably in an amount of 0.5 to 2 weight %; tomatoes (fruit powder, sun dried, granules) preferably in an amount of 2 to 6 weight %; yeast extract preferably in an amount of 0.6 to 2 weight %; zucchini (powder, diced) preferably in an amount of 2 to 8 weight %.

In another refinement, the quinoa-containing minestrone soup includes quinoa, cabbage, potatoes, and rice flour. In still another refinement, the quinoa-containing minestrone soup includes basil, whole leaf, dried preferably in an amount of 0.7 to 2.2 weight %; broccoli powder preferably in an amount of 0.7 to 2.2 weight %; cabbage white (flakes) preferably in an amount of 0.6 to 2.2 weight %; carrot (dehydrated, puffed, powder, pieces) preferably in an amount of 3 to 10 weight %; celeriac preferably in an amount of 2 to 6 weight %; celery seeds powder preferably in an amount of 0.6 to 1.8 weight %; garlic preferably in an amount of 1 to 3 weight %; Onion, white, dried, (powder, minced) preferably in an amount of 3 to 9 weight %; peas preferably in an amount of 3 to 10 weight %; potato preferably in an amount of 6 to 20 weight %; quinoa preferably in an amount of 8 to 23 weight %; rice flour preferably in an amount of 7 to 22 weight %; salt (reg. , kosher, sea salt) preferably in an amount of 2 to 7 weight % (e.g., 0.5 to 2 weight % or 2 to 7 weight %); savoy cabbage preferably in an amount of 3 to 10 weight %; spinach (leaf, powder) preferably in an amount of 0.7 to 2.2 weight %; turmeric preferably in an amount of 0.6 to 1.8 weight %; yeast extract preferably in an amount of 3 to 10 weight %; and zucchini (powder,diced) preferably in an amount of 1 to 5 weight %.

In a refinement, the bean-containing minestrone soup composition includes white beans (e.g., great northern beans), great tea extract, optionally cabbage, and optionally potatoes. In a refinement, the bean-containing minestrone soup composition includes beans (great northern) preferably in an amount of 3 to 10 weight %; cabbage white (flakes) preferably in an amount of 2 to 8 weight %; carrot (dehydrated, puffed, powder, pieces) preferably in an amount of 2 to 8 weight %; celery preferably in an amount of 1 to 4 weight %; green tea extract preferably in an amount of 0.02 to 0.06 weight %; inulin preferably in an amount of 2 to 10 weight %; leeks (granules -10+40) preferably in an amount of 2 to 7 weight %; oil (olive) preferably in an amount of 2 to 7 weight %; onion (powder, minced) preferably in an amount of 2 to 7 weight %; parsley preferably in an amount of 0.2 to 1 weight %; peas preferably in an amount of 3 to 9 weight %; potato preferably in an amount of 15 to 45 weight %; rice flour preferably in an amount of 6 to 18 weight %; salt preferably in an amount of 2 to 8 weight % (e.g., 0.5 to 2 weight % or 2 to 8 weight % ); spinach (leaf, powder) preferably in an amount of 0.5 to 1.5 weight %; tomatoes (fruit powder, sun dried, granules) preferably in an amount of 2 to 7 weight %; and yeast extract preferably in an amount of 0.5 to 1.5 weight %.

In a refinement, the bean-containing minestrone soup composition includes brown beans, carrots, peas, potato, and rice flour. In another refinement, the bean-containing minestrone soup composition includes carrot (dehydrated, puffed, powder, pieces) preferably in an amount of 4 to 14 weight %; celeriac preferably in an amount of 1 to 5 weight %; celery preferably in an amount of 0.5 to 1.6 weight %; leeks preferably in an amount of 2 to 8 weight %; oil (olive) preferably in an amount of 2 to 8 weight %; Onion, white, dried, (powder, minced) preferably in an amount of 3 to 10 weight %; parsley preferably in an amount of 0.5 to 1.5 weight %; peas preferably in an amount of 5 to 18 weight %; potato preferably in an amount of 8 to 24 weight %; rice flour preferably in an amount of 5 to 18 weight %; salt (reg. , kosher, sea salt) preferably in an amount of 4 to 14 weight % (e.g., 1 to 3 weight % or 4 to 14 weight % ); spinach (leaf, powder) preferably in an amount of 0.5 to 1.5 weight %; tomatoes, (fruit powder, sun dried granules) preferably in an amount of 0.9 to 2.8 weight %; turmeric preferably in an amount of 0.3 to 1.2 weight %; and yeast extract preferably in an amount of 0.5 to 1.5 weight %.

In a refinement, the pumpkin soup composition includes pumpkin, green tree extract, optionally rice flour, optionally carrots, and optionally brown rice powder. In a refinement, the pumpkin soup composition includes (new) includes brown rice powder (whole grain) preferably in an amount of 3 to 9 weight %; carrot (dehydrated, puffed, powder, pieces) preferably in an amount of 2 to 8 weight %; green tea extract preferably in an amount of 0.02 to 0.06 weight %; inulin preferably in an amount of 2 to 10 weight %; oil (olive) preferably in an amount of 1 to 7 weight %; onion (powder, minced) preferably in an amount of 1.0 to 3 weight %; pumpkin powder preferably in an amount of 20 to 60 weight %; rice flour preferably in an amount of 15 to 45 weight %; salt preferably in an amount of 2 to 10 weight % (e.g., 0.5 to 2.5 weight % or 2 to 10 weight %); and yeast extract preferably in an amount of 0.3 to 1 weight %.

In a refinement, the first vegetable broth includes carrots, maltodextrin, celery, spinach, and tomatoes. In a refinement, the first vegetable broth includes carrot (dehydrated, puffed, powder, pieces) preferably in an amount of 6 to 18 weight %; celery preferably in an amount of 3 to 10 weight %; garlic preferably in an amount of 3 to 10 weight %; maltodextrin preferably in an amount of 8 to 25 weight %; oil (canola) preferably in an amount of 0.5 to 2 weight %; onion (powder, minced) preferably in an amount of 6 to 18 weight %; parsley preferably in an amount of 3 to 10 weight %; potato preferably in an amount of 1 to 3 weight %; salt preferably in an amount of 0 to 21 weight % (e.g., 1.5 to 5 weight % or 7 to 21 weight % ); spinach (leaf, powder) preferably in an amount of 3 to 10 weight %; tomatoes (fruit powder, sun dried, granules) preferably in an amount of 6 to 18 weight %; and yeast extract preferably in an amount of 1 to 6 weight %.

In a refinement, the second vegetable broth (chicken flavoring) includes carrots, chicken flavoring, maltodextrin, celery, spinach, soy lecithin, and tomatoes. In a refinement, the second vegetable broth composition includes carrot (dehydrated, puffed, powder, pieces) preferably in an amount of 3 to 10 weight %; celery preferably in an amount of 3 to 12 weight %; garlic preferably in an amount of 3 to 9 weight %; maltodextrin preferably in an amount of 8 to 25 weight %; oil (canola) preferably in an amount of 0.5 to 2 weight %; onion preferably in an amount of powder, minced) preferably in an amount of 3 to 12 weight %; parsley preferably in an amount of 3 to 10 weight %; potato preferably in an amount of 1 to 6 weight %; salt preferably in an amount of 8 to 25 weight % (e.g., 2 to 6 weight % or 8 to 25 weight % ); soy lecithin preferably in an amount of 0.5 to 3 weight %; spinach (leaf, powder) preferably in an amount of 3 to 12 weight %; tomatoes (fruit powder, sun dried, granules) preferably in an amount of 6 to 18 weight %; xanthan gum preferably in an amount of 0.5 to 4 weight %; and yeast extract preferably in an amount of 4 to 12 weight %.

In a refinement, the energy drink composition includes glycerin preferably in an amount of 20 to 60 weight %; water (purified) preferably in an amount of 40 to 80 weight %.

In a refinement, the tea composition that includes spearmint includes spearmint leaves organic, preferably in an amount of 70 to 100 weight %.

In a refinement, the tea composition that includes lemon and spearmint includes lemon myrtle organic preferably in an amount of 3 to 12 weight %; lemon peel organic preferably in an amount of 10 to 25 weight %; spearmint leaves organic preferably in an amount of 50 to 95 weight %.

In a refinement, the tea composition that includes hibiscus includes hibiscus tea leaves organic, preferably in an amount of 80 to 100 weight % of the the tea composition.

In a refinement, the algal oil composition includes schizocatrium algae oil (DHA Omega-3), preferably in an amount of 80 to 100 weight % of the the algal oil composition.

In a refinement, the nutrient replenishment composition (NR-1) includes beetroot powder, calcium carbonate, carrots, collard leaf, kale leaf, and tomatoes. In a refinement, the nutrient replenishment composition (NR-1) includes ascorbic acid preferably in an amount of 1 to 3 weight %; beet root powder preferably in an amount of 6 to 20 weight %; beta carotene preferably in an amount of 0.05 to 0.15 weight %; calcium carbonate preferably in an amount of 6 to 20 weight %; carrot (dehydrated, puffed, powder, pieces) preferably in an amount of 6 to 20 weight %; cholecaliciferol preferably in an amount of 0.00 weight %; chromuim Picolinate preferably in an amount of 0.00 weight %; collard leaf powder preferably in an amount of 6 to 20 weight %; cupric sulfate preferably in an amount of 0.01 to 0.06 weight %; cyanocobalamin, 0.00; Dl-alpha tocopherol acetate preferably in an amount of 0.3 to 1 weight %; ferrous fumarate preferably in an amount of 0.2 to 1 weight %; folic acid preferably in an amount of 0.00 weight %; kale leaf preferably in an amount of 6 to 20 weight %; magnesium stearate preferably in an amount of 1 to 6 weight %; manganese sulfate preferably in an amount of 0.04 to 0.08 weight %; niacinamide preferably in an amount of 0.3 to 1 weight %; pantothenic acid preferably in an amount of 0.1 to 0.6 weight %; phytonadione preferably in an amount of 0.00 weight %; potassium iodine preferably in an amount of 0 weight %; pyriodoxine HCI preferably in an amount of 0.03 to 0.1 weight %; riboflavin preferably in an amount of 0.02 to 0.1 weight %; sodium molybdate preferably in an amount of 0.00 weight %; sodium selenate preferably in an amount of 0.00 weight %; spinach (leaf, powder) preferably in an amount of 6 to 20 weight %; thiamine mononitrate preferably in an amount of 0.02 to 0.1 weight %; tomatoes (fruit powder, sun dried, granules) preferably in an amount of 6 to 20 weight %; tribasic calcium phosphate preferably in an amount of 0.5 to 2 weight %; and zinc oxide preferably in an amount of 0.2 to 0.8 weight %.

In a variation, the each of the components of the fasting-mimicking diet package and therefore the fasting-mimicking diet, is substantially gluten free (e.g., each component has less than 20 ppm gluten) or very low gluten (e.g., each component has 20-100 ppm). In other variations, each of the components are provided in a serving size from 20 to 60 g. In other variations, the nut-containing nutrition bar is provided in a serving size from 30 to 60 g; cocoa-containing nutrition bar is provided in a serving size from 15 to 40 g; the olive containing composition (sea salt version) in a serving size from 10 to 20 g; the olive containing composition (garlic version) in a serving size from 10 to 20 g; kale cracker composition is provides in a serving size from 30 to 60 g; In another variation, the kale cracker compositions are provided in a serving size from 20 to 50 g; the vegetable soup compositions are provided in a serving size from 20 to 50 g; the mushroom soup compositions are provided in a serving size from 20 to 50 g; the tomato soup compositions are provided in a serving size from 20 to 50 g; the bean-containing minestrone soup compositions are provided in a serving size from 20 to 50 g; the quinoa-containing minestrone soup compositions are provided in a serving size from 20 to 50 g; the pumpkin soup compositions are provided in a serving size from 20 to 50; the first vegetable both compositions are provided in a serving size from 5 to 15; the second vegetable both compositions are provided in a serving size from 3 to 15; and Energy Drink composition is provided in serving size of 1 to 5 oz.

The following examples illustrate the various embodiments of the present invention. Those skilled in the art will recognize many variations that are within the spirit of the present invention and scope of the claims.

Experiments set forth herein show that the new low sodium ketogenic FMD formulation tested in rats with kidney damage-induced activation of mesenchymal stem cells in comparison to control fed rats. These results strongly support a role for FMD diets, and in particular low sodium diets, in stimulation of kidney regeneration. Furthermore, the proteinuria levels are reduced indicating an improved kidney function.

The formulation of the low sodium ketogenic FMD identified to be effective in rats will be developed for human purposes. Based on the composition above the diet will provide 10% of the normal kcal intake for 5 days. This medical food can be used for response to kidney damage with the exclusion of subjects that they are frail and underweight.

### Methods

Experimental Approach: 12 weeks-old rats were enrolled in either control or diet arms (N=10/group). The diet group is exposed to 6 dietary intervention cycles, biweekly each followed by 10 days refeeding period. This was done at CHLA All rats were maintained in a pathogen-free environment and housed in clear shoebox cages in groups of five animals per cage with constant temperature and humidity and 12 hr/12 hr light/dark cycle and unlimited access to water.

PAN induced nephrosis 40 mg/kg/rat s.c. Early phase proteinuria peaking at 10 days followed by progressive lower-level proteinuria which induces glomerular damage, tubular damage and interstitial fibrosis and inflammation

Diets: The FMD low sodium is a low-calorie and very low protein diet enriched with olive oil-based on human foods. The source of kilocalories is 42% from carbohydrates, 2% protein and 47% fat. Sodium level <1000 mg/day. The diet is mainly composed of vegetable fats and soups. All diet ingredients were thoroughly mixed and then blended together with heated hydrogel (ClearH2O, Maine). To achieve the very low sodium levels, rats were fed a diet containing 10% of normal daily intake on days 1-5.

Statistics and experimental design: All samples represent biological replicates. Unless otherwise specified in figure legends, all center values shown in graphs refer to the mean. Statistical significance of differences among multiple groups (≥ 3) was calculated by performing ANOVA multiple comparisons of the means for each group.

### Results

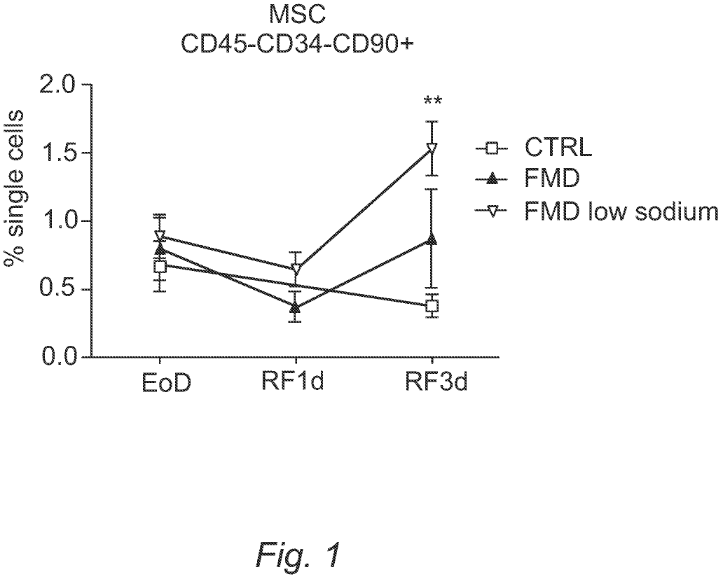

We show results from Sprague Dawley rats subjected to one cycle of FMD, FMD low sodium and ad libitum (CTRL) feeding (FIG. 1). Sprague Dawley rats were injected with puromycin aminonucleoside (PAN) to induce chronic nephrosis. One cycles of FMD have been applied to PAN-induced rats and level of circulating mesenchymal stem cell were analyzed at the end of the diet (EoD), refeeding 1 day (RF1d) and 3 days (RF3d).

We show results from Sprague Dawley rats subjected to 6 cycles of FMD, FMD low sodium, and ad libitum (CTRL) feeding. Sprague Dawley rats were injected with puromycin aminonucleoside (PAN) to induce chronic nephrosis. Multiple cycles of FMD have been applied to PAN-induced rats to determine their effect on renal function and histology. Rats were monitored for renal physiological parameters.

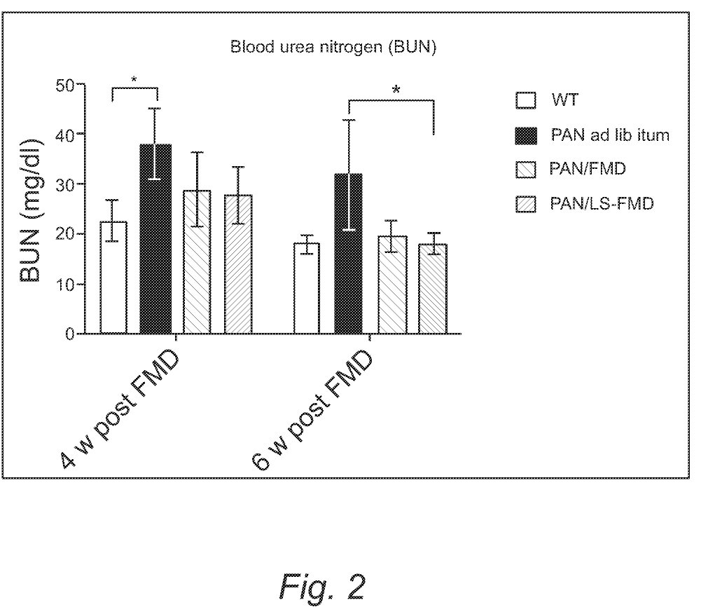

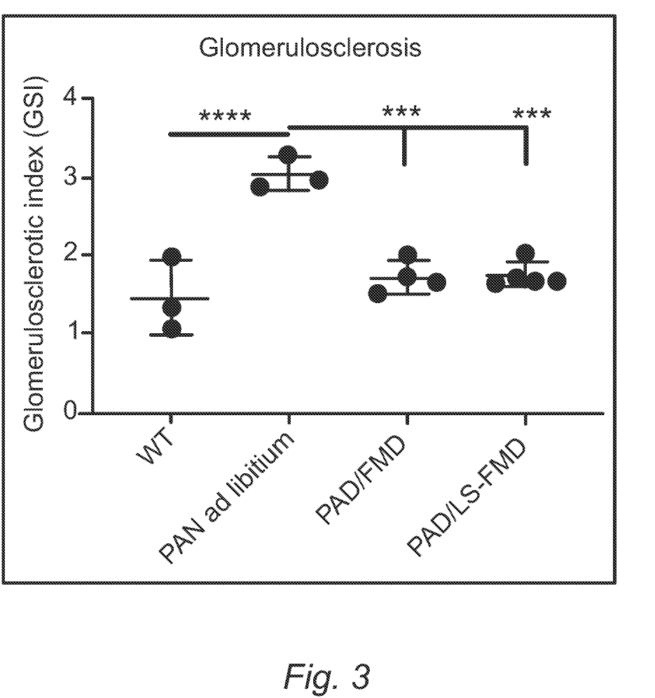

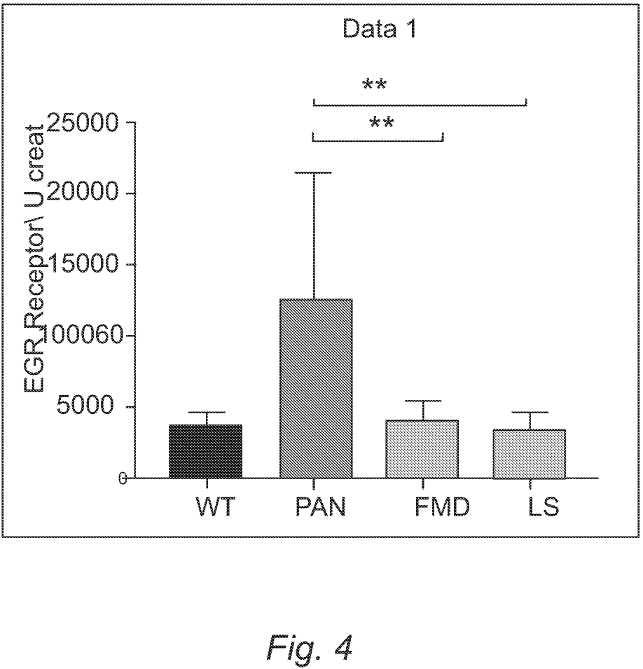

We observed amelioration of proteinuria, BUN and improvement in glomerular histology and stabilization of EGFR in the urine in rats undergoing 6 cycles of FMD and FMD low sodium compared to the PAN group. The effect was sustained long term up to 6 weeks after the last dietary cycle (FIG. 2 and FIG. 3 and FIG. 4).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

References:
1. Levey, A.S. & Coresh, J. Chronic kidney disease. Lancet 379, 165-180 (2012).
2. Raffaghello, L., et al. Starvation-dependent differential stress resistance protects normal but not cancer cells against high-dose chemotherapy. Proc Natl Acad Sci U S A 105, 8215-8220 (2008).
3. Lee, C., et al. Fasting cycles retard growth of tumors and sensitize a range of cancer cell types to chemotherapy. Sci Transl Med 4, 124ra127 (2012).
4. Choi, I.Y., et al. A Diet Mimicking Fasting Promotes Regeneration and Reduces Autoimmunity and Multiple Sclerosis Symptoms. Cell Rep 15, 2136-2146 (2016).
5. Brandhorst, S., et al. A Periodic Diet that Mimics Fasting Promotes Multi-System Regeneration, Enhanced Cognitive Performance, and Healthspan. Cell Metab 22, 86-99 (2015).
6. Cheng, C.W., et al. Prolonged fasting reduces IGF-I/PKA to promote hematopoietic-stem-cell-based regeneration and reverse immunosuppression. Cell Stem Cell 14, 810-823 (2014).

What is claimed is:

1. A method for treating kidney damage comprising:
identifying a subject having kidney damage; and
administering a low sodium fasting-mimicking diet (a low sodium FMD) to the subject for a first time period of 1 to 10 days, the low sodium FMD providing less than 1000 mg of sodium chloride per day of the low sodium FMD, wherein the low sodium FMD includes kilocalories from carbohydrates in an amount from about 30 to 70 percent of the total kilocalories of the low sodium FMD, kilocalories from protein in an amount less than about 10 percent of the total kilocalories of the low sodium FMD, and kilocalories from fat carbohydrates in an amount from about 30 to 70 percent of the total kilocalories of the low sodium FMD.

2. The method of claim 1 wherein the first time period is 5 days.

3. The method of claim 1 wherein the low sodium FMD is administered biweekly.

4. The method of claim 1 wherein the low sodium FMD provides sodium in an amount that less than or equal to 800 mg per day of the low sodium FMD.

5. The method of claim 1 wherein the low sodium FMD provides sodium in an amount that less than or equal to 500 mg per day of the low sodium FMD.

6. The method of claim 1 wherein the low sodium FMD includes kilocalories from carbohydrates in an amount from about 35 to 50 percent of the total kilocalories of the low sodium FMD, kilocalories from protein in an amount from about 0.5 to 10 percent of the total kilocalories of the low sodium FMD, and kilocalories from fat carbohydrates in an amount from about 40 to 60 percent of the total kilocalories of the low sodium FMD.

7. The method of claim 1 wherein the low sodium FMD includes kilocalories from carbohydrates in an amount of about 42 percent of the total kilocalories of the low sodium FMD, kilocalories from protein in an amount of about 2 percent of the total kilocalories of the low sodium FMD, and kilocalories of about fat carbohydrates in an amount from about 47 percent of the total kilocalories of the low sodium FMD.

8. The method of claim 1 wherein the low sodium FMD is periodically or repeatedly administered over a treatment time period.

9. The method of claim 8 wherein the treatment time period is from 1 month to 5 years.

10. The method of claim 1 wherein the low sodium FMD decreases proteinuria and BUN and ameliorates glomerular histology.

11. The method of claim 1 wherein the low sodium FMD re-establishes normal value of EGFR in urine.

12. The method of claim 1 wherein the low sodium FMD provides from 4.5 to 7 kilocalories per pound of subject for a first day and 3 to 5 kilocalories per pound of subject per day for a second to fifth day, the low sodium FMD being a low protein diet that provides less than 28 g of proteins on the first day and less than 18 g of proteins on the second to fifth days.

13. The method of claim 1 wherein the low sodium FMD provides less than 30 g of sugar on the first day; less than 20 g of sugar on the second to fifth days.

14. The method of claim 1 wherein the low sodium FMD provides 20 to 30 grams of monounsaturated fats on the first day.

15. The method of claim 1 wherein the low sodium FMD provides 10 to 15 grams of monounsaturated fats on the second to fifth days.

16. The method of claim 1 wherein the low sodium FMD provides between 6 and 10 grams of polyunsaturated fats on the first day and 3 to 5 grams of polyunsaturated fats on the second to fifth days.

17. The method of claim 1 wherein the low sodium FMD provides less than 12 g of saturated fats on the first day and less than 6 grams of saturated fats on the second to fifth days.

18. The method of claim 1 wherein the low sodium FMD provides 12 to 25 grams of glycerol per day on the second to fifth days.

19. The method of claim 1 wherein the low sodium FMD provides over 5,000 IU of vitamin A per day for days 1-5 and 60-240 mg of vitamin C per day for days 1-5.

20. The method of claim 1 wherein the low sodium FMD provides 400-800 mg of calcium per day for days 1-5; 7.2-14.4 mg of iron per day for days 1-5; 200-400 mg of magnesium per day for days 1-5; 1-2 mg of copper per day for days 1-5; 1-2 mg of manganese per day for days 1-5 and 3.5-7 mcg of Selenium per day for days 1-5.

21. A method for treating glomerulosclerosis comprising:
identifying a subject having glomerulosclerosis; and
administering a low sodium fasting-mimicking diet (a low sodium FMD) to the subject for a first time period of 1 to 10 days, the low sodium FMD providing less than 1000 mg of sodium chloride per day of the low sodium FMD, wherein the low sodium FMD includes kilocalories from carbohydrates in an amount from about 30 to 70 percent of the total kilocalories of the low sodium FMD, kilocalories from protein in an amount less than about 10 percent of the total kilocalories of the low sodium FMD, and kilocalories from fat carbohydrates in an amount from about 30 to 70 percent of the total kilocalories of the low sodium FMD.

22. The method of claim 21 wherein the first time period is 5 days.

23. The method of claim 21 wherein the low sodium FMD is administered biweekly.

24. The method of claim 1 wherein the low sodium FMD provides sodium in an amount that less than or equal to 800 mg per day of the low sodium FMD.

25. The method of claim 1 wherein the low sodium FMD provides sodium in an amount that less than or equal to 500 mg per day of the low sodium FMD.

26. The method of claim 1 wherein the low sodium FMD includes kilocalories from carbohydrates in an amount from about 35 to 50 percent of the total kilocalories of the low sodium FMD, kilocalories from protein in an amount from about 0.5 to 10 percent of the total kilocalories of the low sodium FMD, and kilocalories from fat carbohydrates in an amount from about 40 to 60 percent of the total kilocalories of the low sodium FMD.
